(19) United States
(12) Patent Application Publication
(10) Pub. No.: US 2019/0369086 A1
(43) Pub. Date: Dec. 5, 2019

(54) GAS SENSOR FOR HANDHELD DEVICE

(71) Applicant: TAIWAN CARBON NANO TECHNOLOGY CORPORATION, MIAOLI COUNTY (TW)

(72) Inventors: Yu-Hsuan LIAO, MIAOLI COUNTY (TW); Chia-Pin HUANG, MIAOLI COUNTY (TW); Chun-Hsien TSAI, MIAOLI COUNTY (TW); Ting-Chuan LEE, MIAOLI COUNTY (TW); Chun-Jung TSAI, MIAOLI COUNTY (TW)

(21) Appl. No.: 16/285,572

(22) Filed: Feb. 26, 2019

(30) Foreign Application Priority Data
Jun. 5, 2018 (TW) 107207475

(51) Int. Cl. G01N 33/497

(52) U.S. Cl. CPC G01N 33/497

(57) ABSTRACT

A gas sensor for a handheld device is assembled in a cavity of the handheld device. The gas sensor comprises a sensing chip, a circuit board and a gas channel. During operation, when a to-be-sensed gas is blown to a first sub-channel away from a gas inlet via a second sub-channel in the gas channel adjacent to the gas inlet, a gas pressure is weakened by the channel, so that the gas pressure of the first sub-channel is lower than the gas pressure of the second sub-channel. Therefore, dust and other tiny substances are prevented from entering the handheld device, and the damage to the sensing chip caused by the gas pressure during blowing can also be avoided.

### FIELD OF THE INVENTION

The present invention relates to a gas sensor, in particular to a gas sensor for a handheld device.

### BACKGROUND OF THE INVENTION

Gas sensors are widely used to detect physical properties or chemical properties of gases, and are applied in medical, industrial, environmental protection and other fields.

The related art, such as “portable asthma detection device and stand-alone portable asthma detection device” as mentioned in the Taiwan Publication No. 201219010, relates to a gas sensor which is designed to be applicable in a manner of being externally attached to a handheld device. The gas sensor comprises a housing, a gas detection module including a turbine and a mini airflow sensing component; a gas introducing tube; and a signal delivery unit. In this way, respiratory detection can be performed anytime and anywhere, and the effect of improving the efficiency of monitoring and managing asthma can be achieved.

According to this method, it is possible to detect the gas by the handheld device, however, it is inconvenient and unfavorable to attach the gas sensor to the handheld device.

### SUMMARY OF THE INVENTION

To fulfill said objective, the present invention provides a gas sensor for a handheld device and assembled in a cavity of the handheld device. The gas sensor comprises the following components: a circuit board having at least one through hole; a sensing chip disposed on the circuit board, the sensing chip having at least one sensing region exposed by the through hole; and a gas channel allowing a to-be-sensed gas to enter the through hole and to contact with the sensing region; the gas channel comprises a first sub-channel adjacent to the through hole, a second sub-channel away from the through hole, and a connection channel connecting the first sub-channel and the second sub-channel; wherein the to-be-sensed gas has a first gas pressure P1 while flowing in the first sub-channel and has a second gas pressure P2 while flowing in the second sub-channel, and the gas channel further has a structure that makes P1 lower than P2.

According to the gas sensor for the handheld device, the first sub-channel has a width D₁, the second sub-channel has a width D₂, and D₁ is less than D₂.

According to the gas sensor for the handheld device, the connection channel has an included angle.

According to the gas sensor for the handheld device, the connection channel further comprises a stopper.

According to the gas sensor for the handheld device, a wall of the connection channel further comprises a pressure relief hole.

According to the gas sensor for the handheld device, the sensing region comprises at least two sensing components for detecting different target molecules.

According to the gas sensor for the handheld device, the circuit board is a printed circuit board.

According to the gas sensor for the handheld device, the sensing chip is a flip chip.

The gas sensor for the handheld device further comprises a waterproof breathable membrane disposed between the sensing region and the gas channel.

The gas sensor for the handheld device further comprises a barrier layer which covers one side of the sensing chip and the circuit board opposite to the waterproof breathable membrane.

In summary, the present invention has the following features compared with the prior art:

1. the gas sensor is integrated in the handheld device, particularly in the cavity of the handheld device, e.g., in a cavity capable of accommodating a microphone, but is not limited thereto. The gas sensor can be integrated not only with the existing process of the handheld device but also conveniently for the user to use, without the need to carry other sensors additionally as in the prior art.

2. Through the design of the gas channel, the present invention reduces the gas pressure during blowing without damaging the sensing chip.

3. In an embodiment of the present invention, the gas sensor is provided with the waterproof breathable membrane to block moisture, dust and other tiny substances, so as not to damage the handheld device, and so as to prevent the sensing chip from being inaccurate due to moisture disturbance.

### BRIEF DESCRIPTION OF THE DRAWINGS

FIG. 1 is a schematic diagram of a first embodiment of a gas sensor of the present invention;

FIG. 2 is a schematic diagram of a second embodiment of the gas sensor of the present invention;

FIG. 3 is a schematic diagram of a third embodiment of the gas sensor of the present invention;

FIG. 4 is a schematic diagram of a fourth embodiment of the gas sensor of the present invention; and

FIG. 5 is a schematic diagram of the gas sensor of the present invention assembled on the handheld device.

### DETAILED DESCRIPTION OF THE PREFERRED EMBODIMENTS

The detailed description and technical content related to the present invention will now be described as follows:

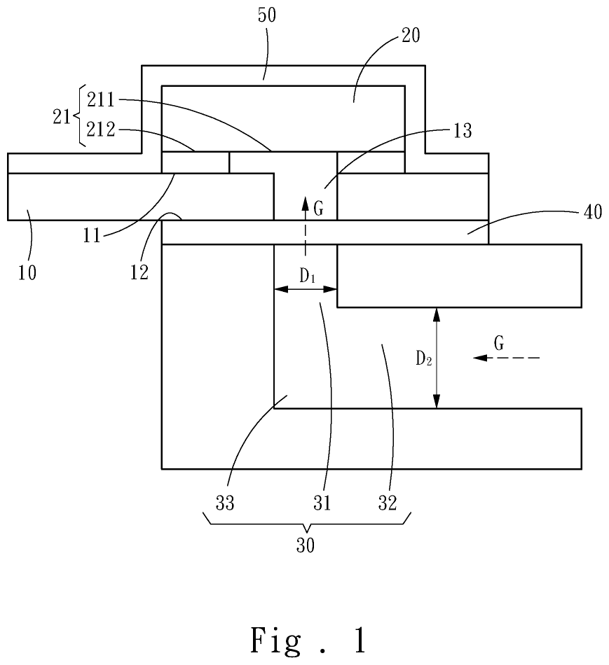

Referring to FIGS. 1, 2, 3, 4, and 5, the present invention relates to a gas sensor applied to a handheld device M. The gas sensor mainly comprises a circuit board 10, a sensing chip 20, and a gas channel 30.

Referring to FIG. 1, FIG. 1 is a schematic diagram of a first embodiment of the gas sensor of the present invention. The circuit board 10 has an upper surface 11, a lower surface 12 and a through hole 13 that penetrates through the upper surface 11 and the lower surface 12. The upper surface 11 of the circuit board 10 is used for disposing the sensing chip 20. In this embodiment, the circuit board 10 is a printed circuit board, and the lower surface 12 is a waterproof breathable membrane attaching region for attaching a waterproof breathable membrane 40. The waterproof breathable membrane 40 covers the through hole 13 to provide a better waterproof and dustproof effect.

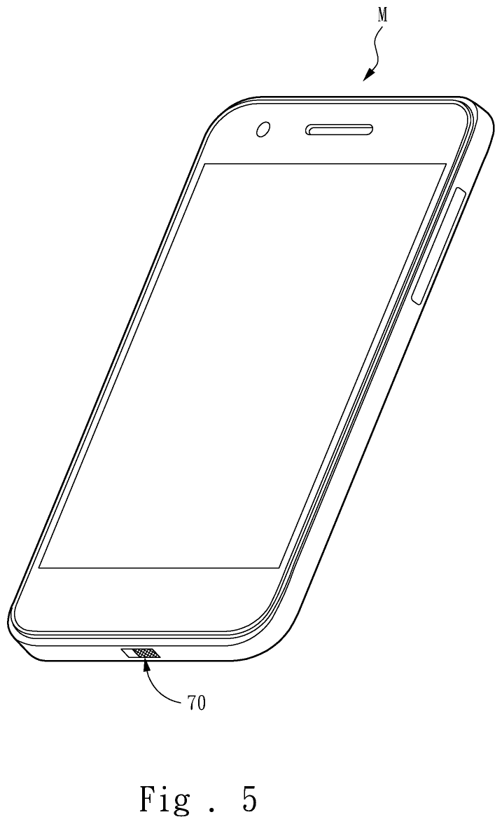

The sensing chip 20 has a sensing surface 21 on which at least one bonding region 212 and at least one sensing region 211 which is in contact with a to-be-sensed gas G are formed. In an embodiment, the sensing region 211 is formed by a sensing film which is fabricated using an ultra-low concentration sensing film coating technology. In an embodiment, the sensing chip 20 may be a flip chip. FIG. 5 is a schematic diagram of the gas sensor of the present invention assembled on the handheld device. The handheld device M comprises a cavity 70 for accommodating a microphone. The cavity 70 of the microphone is usually disposed at the bottom of the handheld device M as needed for use. When the flip chip is used to invert the sensing chip 20 relative to the circuit board 10 and to expose the sensing region 211 by the through hole 13 of the circuit board 10 below, the gas sensor is adapted to be assembled in the cavity 70, in which the microphone is accommodated, of the handheld device M. Further, the structure of the flip chip can provide a shorter circuit, which can greatly reduce signal interference and reduce the loss of circuit connection.

The gas channel 30 is communicated with the sensing region 211 via the through hole 13. The gas channel 30 comprises a first sub-channel 31 adjacent to the through hole 13, a second sub-channel 32 away from the through hole 13, and a connection channel 33 connecting the first sub-channel 31 and the second sub-channel 32. The to-be-sensed gas G has a first gas pressure P1 while flowing in the first sub-channel 31 and has a second gas pressure P2 while flowing in the second sub-channel 32, and the gas channel 30 further has a structure that makes the first gas P1 lower than the second gas pressure P2. In this embodiment, this structure is a right-angle bend by which the to-be-sensed gas G is first blocked by the wall at the connection channel 33 after entering the gas channel 30 and moves upward, and finally passes through the waterproof breathable membrane 40 and reaches the sensing region 211. As such, the first gas pressure P1 is lower than the second gas pressure P2. Therefore, the structure of the sensing region 211 is prevented from being damaged by the stress of the waterproof breathable membrane 40 after repeated use. In other embodiments, the first sub-channel 31 has a width D₁, the second sub-channel 32 has a width D₂, and D₁ is less than D₂, e.g., D₂=2D₁.

In other embodiments of the present invention, the gas sensor further comprises a barrier layer 50 which covers one side of the sensing chip 20 and the circuit board 10 opposite to the waterproof breathable membrane 40. The barrier layer 50 isolates the sensing chip 20 and the circuit board 10 from the handheld device M to prevent a signal of the sensing chip 20 from interfering with the handheld device M so as not to affect the use of the handheld device M.

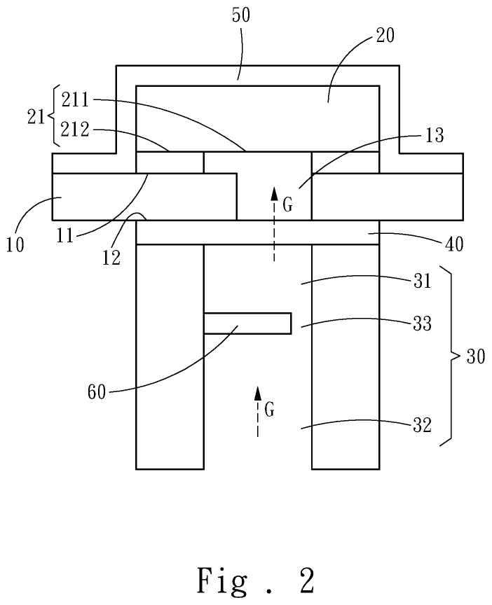

Referring to FIG. 2 for the second embodiment, the connection channel 33 further comprises a stopper 60, such that when the to-be-sensed gas G enters from the second sub-channel 32, the second gas pressure P2 is reduced via the stopper 60, so that P1 is lower than P2. In the case of the second embodiment, the connection channel 33 may or may not have an included angle, and the stopper 60 may be one or plural.

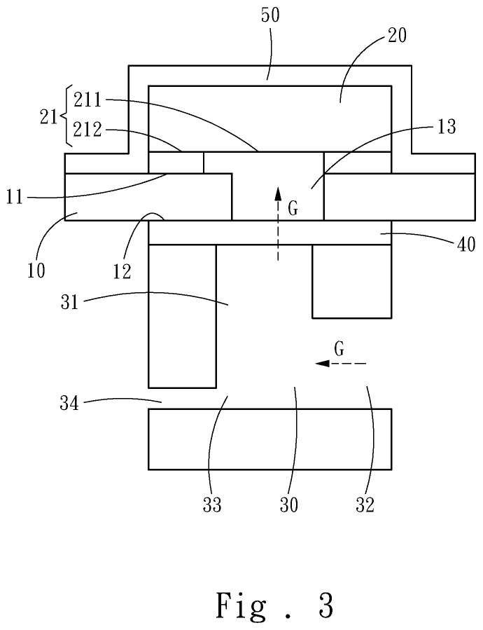

Alternatively, in the third embodiment as shown in FIG. 3, a pressure relief hole 34 may be further formed in a wall of the connection channel 33. During the operation, part of the to-be-sensed gas G will be discharged from the gas channel 30 via the pressure relief hole 34, and only the other part of the to-be-sensed gas G can smoothly enter the first sub-channel 31, such that the effect of reducing the second gas pressure P₂ to make P₁ lower than P₂ can also be achieved.

Based on the above structure, the to-be-sensed gas G will pass through the gas channel 30 along a direction of the gas channel 30 (as indicated by the arrow) during the operation, and finally pass through the through hole 13 to reach a position of the sensing chip 20, thereby the composition of the to-be-sensed gas G can be sensed.

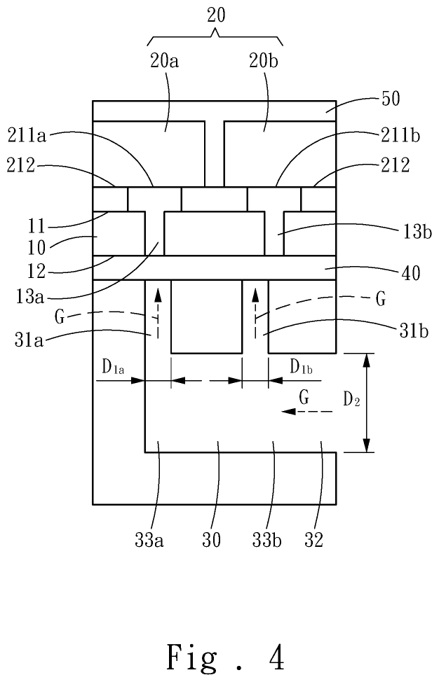

Referring to FIG. 4, the present invention further achieves the effect of detecting a plurality of different gases by comprising at least two sensing components 20a, 20b in the sensing region 211 for detecting different target molecules. For example, as shown in FIG. 4, the sensing chip 20 is provided with sensing components 20a, 20b for detecting particular molecules, and the sensing components 20a, 20b are exposed to the sensing regions 211a, 211b and are in contact with the to-be-sensed gas G respectively by the through holes 13a, 13b. The widths D₁ₐ, D_{1b} of the first sub-channels 31a, 31b are less than the width D₂ of the second sub-channel 32. However, the above description is merely an example, and the present invention is not limited thereto.

In summary, the gas sensor for the handheld device M is built in the handheld device M, which is different from the prior art in which the sensor is externally attached to the handheld device M, such that the convenience, stability and dexterity can be improved. Therefore, the gas sensor of the present invention is expected to be widely used by the public.

What is claimed is:

1. A gas sensor for a handheld device, characterized in that the gas sensor is assembled in a cavity of the handheld device and comprises:
a circuit board comprising at least one through hole;
a sensing chip disposed on the circuit board, the sensing chip comprising at least one sensing region exposed by the through hole; and
a gas channel allowing a to-be-sensed gas to enter the through hole and to contact with the sensing region, the gas channel comprising a first sub-channel adjacent to the through hole, a second sub-channel away from the through hole, and a connection channel connecting the first sub-channel and the second sub-channel;
wherein the to-be-sensed gas has a first gas pressure P1 while flowing in the first sub-channel and has a second gas pressure P2 while flowing in the second sub-channel, and the gas channel comprises a structure to make P1 lower than P2.

2. The gas sensor for the handheld device according to claim 1, wherein the first sub-channel has a width D₁, the second sub-channel has a width D₂, and D₁ is less than D₂.

3. The gas sensor for the handheld device according to claim 1, wherein the connection channel has an included angle.

4. The gas sensor for the handheld device according to claim 1, wherein the connection channel further comprises a stopper.

5. The gas sensor for the handheld device according to claim 1, wherein a wall of the connection channel comprises a pressure relief hole.

6. The gas sensor for the handheld device according to claim 1, wherein the sensing region comprises at least two sensing components for detecting different target molecules.

7. The gas sensor for the handheld device according to claim 1, wherein the circuit board is a printed circuit board.

8. The gas sensor for the handheld device according to claim 1, wherein the sensing chip is a flip chip.

9. The gas sensor for the handheld device according to claim 1, wherein the gas sensor further comprising a waterproof breathable membrane disposed between the sensing region and the gas channel.

10. The gas sensor for the handheld device according to claim 9, wherein the gas sensor further comprising a barrier layer which covers one side of the sensing chip and the circuit board opposite to the waterproof breathable membrane.
